(19) United States
(12) Patent Application Publication
(10) Pub. No.: US 2019/0328816 A1
(43) Pub. Date: Oct. 31, 2019

(54) Chinese medicinal composition that expels parasitic Toxoplasma gondii and a method of preparing the same

(71) Applicant: Zhuhai Hantang Biotechnology Engineering Co., Ltd., Zhuhai (CN)

(72) Inventor: Huiqiu CHEN, Hunan (CN)

(21) Appl. No.: 16/503,650

(22) Filed: Jul. 5, 2019

(51) Int. Cl. A61K 36/736; A61K 36/282; A61K 36/288; A61K 36/88; A61K 36/076; A61K 36/185; A61K 36/8945; A61K 36/47; A61P 33/02; A61K 9/19

(52) U.S. Cl. CPC A61K 36/736; A61K 36/282; A61K 36/288; A61K 36/88; A61K 9/19; A61K 36/185; A61K 36/8945; A61K 36/47; A61P 33/02; A61K 36/076

(57) ABSTRACT

A Chinese medicinal composition that expels parasitic Toxoplasma gondii, including the following ingredients: Mume fructus 5-10 parts by weight, Artemisiae annuae herba 50-60 parts by weight, Taraxaci herba 5-10 parts by weight, Polygonati rhizoma 10-15 parts by weight, Poria 5-10 parts by weight, Angelicae dahuricae radix 5-8 parts by weight, Dioscoreae rhizoma 5-15 parts by weight, and Euphorbia humifusa 5-10 parts by weight. A method of preparing the Chinese medicinal composition is also disclosed.

A Chinese medicinal composition that expels parasitic Toxoplasma gondii and a method of preparing the same

### BACKGROUND OF THE INVENTION

The present invention relates to the technical field of Chinese medicine, and more specifically relates to a Chinese medicinal composition that expels parasitic Toxoplasma gondii and a method of preparing the same.

Toxoplasmosis is a kind of human and non-human animal infection caused by Toxoplasma gondii. Toxoplasma gondii is a kind of parasite belonging to a type of protozoan organism which has the smallest size and a simple structure. Cats and other animals from the cat family are definitive hosts of Toxoplasma gondii. Toxoplasma gondii lives parasitically in the epithelial cells of the small intestines of these animals, forming cystozygotes excreted together with feces, which are then eaten by other mammals and birds who then become infected, and the cystozygotes are developed into cysts in the tissues of these mammals and birds. Cysticzygotes and cysts are different forms of the Toxoplasma gondii in different developmental stages. Although Toxoplasma gondii is not “picky”, if not living in the definitive hosts, Toxoplasma gondii can only perform asexual reproduction in the bodies of other animals, and its offspring cannot be spread to the outside world.

Nowadays, checks for Toxoplasma antibodies in pregnant women are common in many hospitals. In the prior art, spiramycin is commonly used in pharmaceutical treatment for Toxoplasma infection in pregnant women because spiramycin is a macrolide which is safe for pregnant women due to its low toxicity, however, a large dose of spiramycin may cause gastrointestinal reactions such as nausea, vomiting, and diarrhea. Also, this treatment method can only kill the Toxoplasma gondii partially and temporarily, but cannot inhibit the reproductive activity of Toxoplasma gondii and the Toxoplasma cysts, so it is very likely that Toxoplasma infection may recur.

### BRIEF SUMMARY OF THE INVENTION

In view of the aforesaid disadvantages now present in the prior art, the present invention provides a Chinese medicinal composition that expels parasitic Toxoplasma gondii. The Chinese medicinal composition can inhibit the reproductive activity of Toxoplasma gondii, reduce its activeness and thus gradually expel Toxoplasma gondii out of the bodies of humans or other non-human animals.

To fulfil the above objects, the present invention provides the following technical solutions: A Chinese medicinal composition that expels parasitic Toxoplasma gondii, comprising the following ingredients in parts by weight: Mume fructus (Smoked Plum) 5-10 parts by weight, Artemisiae annuae herba (Sweet Wormwood Herb) 25-30 parts by weight, Taraxaci herba (Mongolian Dandelion Herb) 5-10 parts by weight, Polygonati rhizoma (Siberian Solomonseal Rhizome) 10-15 parts by weight, Poria (Indian Buead Tuckahoe) 5-10 parts by weight, Angelicae dahuricae radix (Taiwan Angelica Root) 5-8 parts by weight, Dioscoreae rhizoma (Common Yan Rhizome) 5-15 parts by weight, and Euphorbia humifusa (Humifuse Euphorbia Herb) 5-10 parts by weight.

Specifically, the ingredients comprise: Mume fructus (Smoked Plum) 6-8 parts by weight, Artemisiae annuae herba (Sweet Wormwood Herb) 50-60 parts by weight, Taraxaci herba (Mongolian Dandelion Herb) 5-8 parts by weight, Polygonati rhizoma (Siberian Solomonseal Rhizome) 12-15 parts by weight, Poria (Indian Buead Tuckahoe) 8-10 parts by weight, Angelicae dahuricae radix (Taiwan Angelica Root) 6-8 parts by weight, Dioscoreae rhizoma (Common Yan Rhizome) 10-12 parts by weight, and Euphorbia humifusa (Humifuse Euphorbia Herb) 8-10 parts by weight.

More specifically, the ingredients comprise: Mume fructus (Smoked Plum) 8 parts by weight, Artemisiae annuae herba (Sweet Wormwood Herb) 28 parts by weight, Taraxaci herba (Mongolian Dandelion Herb) 8 parts by weight, Polygonati rhizoma (Siberian Solomonseal Rhizome) 15 parts by weight, Poria (Indian Buead Tuckahoe) 10 parts by weight, Angelicae dahuricae radix (Taiwan Angelica Root) 6 parts by weight, Dioscoreae rhizoma (Common Yan Rhizome) 10 parts by weight, and Euphorbia humifusa (Humifuse Euphorbia Herb) 8 parts by weight.

The present invention also provides a method of preparing said Chinese medicinal composition that expels parasitic Toxoplasma gondii, comprising the following steps:

S1: weighing the ingredients according to the pre-determined parts by weight, cleaning the ingredients, and then drying the ingredients;

S2: mixing the dried ingredients together, and crushing the dried ingredients into granules, adding water to the granules, and then boiling the granules to obtain a concentrated solution;

S3: performing supercritical extracting of the concentrated solution using CO₂ as an extracting agent, thereby obtaining an extract;

S4: spray drying the extract to obtain powder of the extract; the powder is said Chinese medicinal composition that expels parasitic Toxoplasma gondii.

In S2, a ratio of concentration of the concentrated solution is 20:1.

In S3, transferring the concentrated solution into a SEF (supercritical extraction fluid) production apparatus, adding distilled water into the SEF production apparatus, and then performing said supercritical extracting of the concentrated solution at a temperature under 25° C.

In S4, said spray drying comprises the following steps:

1: sterilizing the extract;

2: concentrating the extract under a vacuum condition to remove 75%-85% of water in the extract;

3: spraying the concentrated extract as atomized particles by using an atomizer, and then drying the concentrated extract to obtain the powder of the extract under centrifugal force;

wherein said process of sterilizing the extract comprises heating the extract at a temperature of 90-100° C. for 10-15 minutes.

The present invention has the following beneficial effects: the Chinese medicinal composition that expels parasitic Toxoplasma gondii can effectively inhibit the reproduction activity of parasitic Toxoplasma gondii, and gradually expel Toxoplasma gondii completely from bodies of human or non-human animals. Said Chinese medicinal composition is non-toxic and has no side effects, instead, it helps improve well-being of cells, improve the general health of human bodies, repair the immune systems damaged by Toxoplasma infection, and eventually allow human bodies to get rid of diseases induced by Toxoplasma infection through their self-regulating and defense systems.

### DETAILED DESCRIPTION OF THE INVENTION

The present invention is further described in detail below with reference to an embodiment. It should be noted that, the present invention should not be limited to the embodiment described herein, but instead can be embodied in different ways.

The embodiment provided below is only intended to provide a clearer and more thorough understanding of the technical disclosure of the present invention.

A Chinese medicinal composition that expels parasitic Toxoplasma gondii, comprising the following ingredients in parts by weight: Mume fructus (Smoked Plum) 8 parts by weight, Artemisiae annuae herba (Sweet Wormwood Herb) 28 parts by weight, Taraxaci herba (Mongolian Dandelion Herb) 8 parts by weight, Polygonati rhizoma (Siberian Solomonseal Rhizome) 15 parts by weight, Poria (Indian Buead Tuckahoe) 10 parts by weight, Angelicae dahuricae radix (Taiwan Angelica Root) 6 parts by weight, Dioscoreae rhizoma (Common Yan Rhizome) 10 parts by weight, and Euphorbia humifusa (Humifuse Euphorbia Herb) 8 parts by weight.

The ingredients comprise purely Chinese medicine in a selected appropriate proportion, so as to achieve a balanced use of the ingredients free of side effects.

The present invention also provides a method for preparing the Chinese medicinal composition that expels parasitic Toxoplasma gondii, comprising the following steps:

S1: weighing the ingredients according to said parts by weight of each ingredient, cleaning the ingredients by using pure water, and then drying the ingredients in a natural condition;

S2: mixing the dried ingredients together, and crushing the dried ingredients into granules by using a crusher, adding water to the granules, and then boiling the granules to obtain a concentrated solution, wherein a ratio of concentration of the concentrated solution is 20:1;

S3: transferring the concentrated solution into a SEF (supercritical extraction fluid) production apparatus, adding distilled water into the SEF production apparatus, and then performing supercritical extracting of the concentrated solution at a temperature under 25° C. using CO₂ as an extracting agent, thereby obtaining an extract;

S4: spray drying the extract to obtain powder of the extract; the powder is said Chinese medicinal composition that expels parasitic Toxoplasma gondii.

In S4, said spray drying comprises the following steps:

1: sterilizing the extract by heating the extract at a temperature of 95° C. for 12 minutes;

2: concentrating the extract under a vacuum condition to remove 75%-85% of water in the extract;

3: spraying the concentrated extract as atomized particles by using an atomizer, and then drying the concentrated extract to obtain the powder of the extract under centrifugal force.

The Chinese medicinal composition eventually obtained has a powder form. It is suggested that the Chinese medicinal composition be packaged and vacuumed using sterilized plastic bags so as to prolong the storage period of the Chinese medicinal composition. Consume the Chinese medicinal composition with warm water.

In the embodiment, the SEF production apparatus is an ordinary apparatus for extraction according to prior art, and therefore will not be described in detail herein.

The method of preparing the Chinese medicinal composition according to the present invention maintains as much as possible the pharmaceutically value of the ingredients so as to increase the efficacy of the Chinese medicinal composition. Reproduction activity of Toxoplasma gondii can be effectively inhibited after consuming continuously the Chinese medicinal composition of the present invention for more than 1 month, and Toxoplasma gondii can be gradually expelled completely from bodies of human or non-human animals. Said Chinese medicinal composition is non-toxic and has no side effects, instead, it helps improve well-being of cells, improve the general health of human bodies, repair the immune systems damaged by Toxoplasma infection, and eventually allow human bodies to get rid of diseases induced by Toxoplasma infection through their self-regulating and defense systems.

It should be noted that, said Chinese medicinal composition is not suitable for child and infant, and should not be consumed with strong tea or other health preservation Chinese medicine considered having a warm (wen) nature according to the concept of “qi” in Chinese medicine.

The embodiment described above is only a preferred embodiment of the present invention, and should not limit the scope of the present invention. Any changes or modifications made by a person skilled in this field of art based on the teachings of the present invention without deviating from the concept of the present invention should also fall within the scope of protection defined by the claims of the present invention.

What is claimed is:

1. A Chinese medicinal composition that expels parasitic Toxoplasma gondii, comprising the following ingredients in parts by weight: Mume fructus 5-10 parts by weight, Artemisiae annuae herba 50-60 parts by weight, Taraxaci herba 5-10 parts by weight, Polygonati rhizoma 10-15 parts by weight, Poria 5-10 parts by weight, Angelicae dahuricae radix 5-8 parts by weight, Dioscoreae rhizoma 5-15 parts by weight, and Euphorbia humifusa 5-10 parts by weight.

2. The Chinese medicinal composition of claim 1, wherein the ingredients comprise: Mume fructus 6-8 parts by weight, Artemisiae annuae herba 25-30 parts by weight, Taraxaci herba 5-8 parts by weight, Polygonati rhizoma 12-15 parts by weight, Poria 8-10 parts by weight, Angelicae dahuricae radix 6-8 parts by weight, Dioscoreae rhizoma 10-12 parts by weight, and Euphorbia humifusa 8-10 parts by weight.

3. The Chinese medicinal composition of claim 2, wherein the ingredients comprise: Mume fructus 8 parts by weight, Artemisiae annuae herba 28 parts by weight, Taraxaci herba 8 parts by weight, Polygonati rhizoma 15 parts by weight, Poria 10 parts by weight, Angelicae dahuricae radix 6 parts by weight, Dioscoreae rhizoma 10 parts by weight, and Euphorbia humifusa 8 parts by weight.

4. A method for preparing said Chinese medicinal composition that expels parasitic Toxoplasma gondii in claim 1, comprising the following steps:
S1: weighing the ingredients according to pre-determined parts by weight of each ingredient, cleaning the ingredients, and then drying the ingredients;
S2: mixing the dried ingredients together, and crushing the dried ingredients into granules, adding water to the granules, and then boiling the granules to obtain a concentrated solution;
S3: performing supercritical extracting of the concentrated solution using CO₂ as an extracting agent, thereby obtaining an extract;
S4: spray drying the extract to obtain powder of the extract; the powder is said Chinese medicinal composition that expels parasitic Toxoplasma gondii.

5. The method of claim 4, wherein in S2, a ratio of concentration of the concentrated solution is 20:1.

6. The method of claim 4, wherein in S3, transferring the concentrated solution into a supercritical extraction fluid production apparatus, adding distilled water into the SEF production apparatus, and then performing said supercritical extracting of the concentrated solution at a temperature under 25° C.

7. The method of claim 4, wherein in S4, said spray drying comprises the following steps:
1: sterilizing the extract;
2: concentrating the extract under a vacuum condition to remove 75%-85% of water in the extract;
3: under centrifugal force, spraying the concentrated extract as atomized particles by using an atomizer, and then drying the concentrated extract to obtain the powder of the extract;

8. The method of claim 7, wherein said process of sterilizing the extract comprises heating the extract at a temperature of 90-100° C. for 10-15 minutes.
